(19) United States
(12) Patent Application Publication
(10) Pub. No.: US 2012/0088954 A1
(43) Pub. Date: Apr. 12, 2012

(54) Cardiac Pump

(75) Inventor: Graham Foster, Swansea (GB)

(73) Assignee: CALON CARDIO TECHNOLOGY LTD, Swansea (GB)

(21) Appl. No.: 13/264,284

(22) PCT Filed: Apr. 19, 2010
(86) PCT No.: PCT/GB2010/000778

§ 371 Date: Nov. 30, 2011

(30) Foreign Application Priority Data
Apr. 17, 2009 (GB) 0906642.4

(51) Int. Cl. A61M 1/10

(52) U.S. Cl. 600/16

(57) ABSTRACT

The pump is of an axial flow rotary pump, suitable for implantation into the human heart or vascular system, and comprises an elongate tubular casing (1,2) defining an inlet (4) for blood, an outlet (5) for blood longitudinally spaced from the inlet, and a primary substantially axial blood flow path (6) along the interior of the casing from the inlet to the outlet, the casing including an electric motor stator (7). There is an elongate rotatable element (3) arranged to fit within the casing with spacing between an outer surface of the rotatable element and an inner surface of the casing. The tubular rotatable element comprises an electric motor rotor portion (10) arranged to be driven by the electric motor stator and a rotary impeller (11) for impelling blood along the blood flow path. The casing is formed as an upstream tubular member (2) having an open front end, and a downstream tubular member (1) having open front and rear ends, the upstream tubular member including the stator, and the downstream tubular member, which encircles the impeller, having a rear end fitted to the upstream tubular member in fluid tight manner.

The present invention concerns miniaturised cardiac pumps suitable for implantation into the human heart or vascular system

Heart Failure is major global health problem resulting in many thousands of deaths each year. Until recently the only way to curatively treat advanced stage heart failure has been by heart transplant or the implantation of a total mechanical heart. Unfortunately donor hearts are only able to meet a tiny fraction of the demand and total mechanical hearts have yet to gain widespread acceptance due to the technical difficulties involved with these devices.

Ventricle assist devices (VADs) have been gaining increased acceptance over the last three decades primarily as a bridge to transplant devices. The devices are implanted long term and work alongside a diseased heart to boost its output and keep the patient alive and/or give a better quality of life whilst awaiting transplant. The use of these devices has had an unexpected result in some patients: the reduction in strain on the heart over a period of time has led to significant spontaneous recovery of the left ventricle. This gives hope to many patients for whom a donor heart may not become available as it could be the case that the early implantation of a VAD may allow their condition to recover before the disease reaches the most advanced stages. It is also a far more preferable outcome to have ones own heart recover than undergo a transplant even if donor hearts are available.

At present, one of the main reasons preventing VADs from being fitted on a more routine basis is the highly invasive surgical procedure required to fit the devices. Typically a sternotomy, full heart lung bypass, and major procedures to the heart and thoracic aorta are required to fit a VAD. Presently the expense and risk of such an operation cannot be justified except in the case of those in the most advanced stages of Heart Failure. If the long term implantation of a VAD or an equivalent circulatory assist device (CAD) could be achieved with a less invasive surgical procedure, ideally eliminating the need for a sternotomy and heart lung bypass, then the use of CADs to treat heart failure in its earlier stages could become far more widespread and routine.

The key to a less invasive implantation procedure for a CAD is to make the device as small as possible so that it can be implanted using a ‘keyhole’ type procedure that eliminates the need for the above invasive surgery.

The other main reason preventing widespread use of CADs is the high cost of existing devices. Generally, highly specialised materials and manufacturing processes are employed to manufacture these devices resulting in a very costly end product.

As a result of the above considerations, there exists a need to develop miniaturised cardiac pumps suitable for implantation into the human heart or vascular system, which can permit low cost manufacture.

It is desirable to provide such a pump that is suitable for minimally invasive implantation into the human heart or vascular system, and can be manufactured by low cost production methods.

Known types of axial flow rotary pump suitable for implantation into the human heart or vascular system comprise, in general,

an elongate tubular casing defining an inlet for blood, an outlet for blood longitudinally spaced from the inlet and a substantially axial blood flow path from the inlet to the outlet along the interior of the casing, the casing including an electric motor stator,
an elongate rotatable element arranged to fit within the casing with spacing between an outer surface of the rotatable element and an inner surface of the casing, the tubular rotatable element comprising an electric motor rotor portion arranged to be driven by the electric motor stator, and
a rotary impeller for impelling blood from the inlet to the outlet.

Typically, such a pump would reside in the left ventricle of the heart and would operate as a left ventricle assist device (LVAD), although it may be adapted to support other chambers of the heart. An example of such a pump is an axial flow rotary pump powered by an integrated electric motor

According to the invention, the casing is formed from an upstream (rear) tubular member having an open front end, and a downstream (front) tubular member having open front and rear ends, the upstream tubular member including the stator, and the downstream tubular member, which encircles the impeller, having a rear end fitted to (and preferably within) the upstream tubular member. Preferred features of the cardiac pump are defined in the accompanying claims.

The fit between the rear end of the downstream tubular member and the upstream tubular member should be such that there is essentially no fluid path between the two tubular members and minimal lines, sharp edges or other disturbances to blood flow.

It is preferred that each of the upstream tubular element and the downstream tubular element, and optionally also the rotatable element, each comprises a selected physiologically acceptable, sterilisable, mouldable engineering plastics material, such as a polyether ether ketone (PEEK) or a high performance polyamide. Other mouldable materials, such as biocompatible ceramics or metals may alternatively be employed. It is especially preferred that each of the upstream tubular element and the downstream tubular element is a unitary moulding, and it is also preferred that each of the tubular elements has a longitudinal axis of symmetry and/or is free of moulding undercuts. The materials of each of the downstream tubular element, the upstream tubular element and the rotatable element may be the same or different.

The upstream tubular member is preferably formed as a unitary moulding by a process known as overmoulding, in which the motor stator is encapsulated within the mouldable material as described above.

It is preferred that the upstream tubular member has a mouth at its front end, the mouth being shaped to receive the rear end of the downstream tubular member. The downstream tubular member may be a slide fit into that mouth, or the mouth may have formations for complementary engagement with corresponding formations around the circumference of the rear end of the downstream tubular member, such that, for example, they may be a press-fit or snap-fit into one another. Especially in this latter embodiment, it is preferred that the downstream tubular element should have a circumferential collar, to inhibit over-insertion thereof.

It is preferred that the mouth at the front end of the upstream tubular member is of greater diameter than an opening at the rear end of the upstream tubular member. It is further preferred that the mouth has an outer diameter greater than an outer diameter of the rear end of the upstream tubular member. This feature can permit the upstream tubular member to be formed as a unitary moulding (overmoulded around the stator as described above) in a two part mould, free of undercuts.

It is further preferred that the upstream tubular member has a series of circumferentially spaced inlets for blood around the periphery thereof. Such inlets may separated from one another by a series of longitudinally extending ribs, which preferably extend from upstream of the inlets to downstream thereof. It is further preferred that such ribs are provided with a mechanical reinforcement which extends substantially around the circumference of the upstream tubular member.

In a further preferred embodiment of the present invention, the rotatable element may be provided with a circumferentially extending surface which seats on a complementary circumferential surface towards the mouth of the upstream tubular member. The complementary surfaces may be, for example, approximately perpendicular to the axis of the rotatable element, or at an obtuse angle (that is, greater than 90°, but less than 180° to the axis of the rotatable element). The complementary surfaces may be provided with suitable bearing elements, as will be described below with reference to the embodiments illustrated in the accompanying drawings.

Embodiments of the present invention, and preferred features thereof, will now be described in more detail, with reference to accompanying drawings, in which like parts are denoted by like reference numerals throughout. In the drawings:

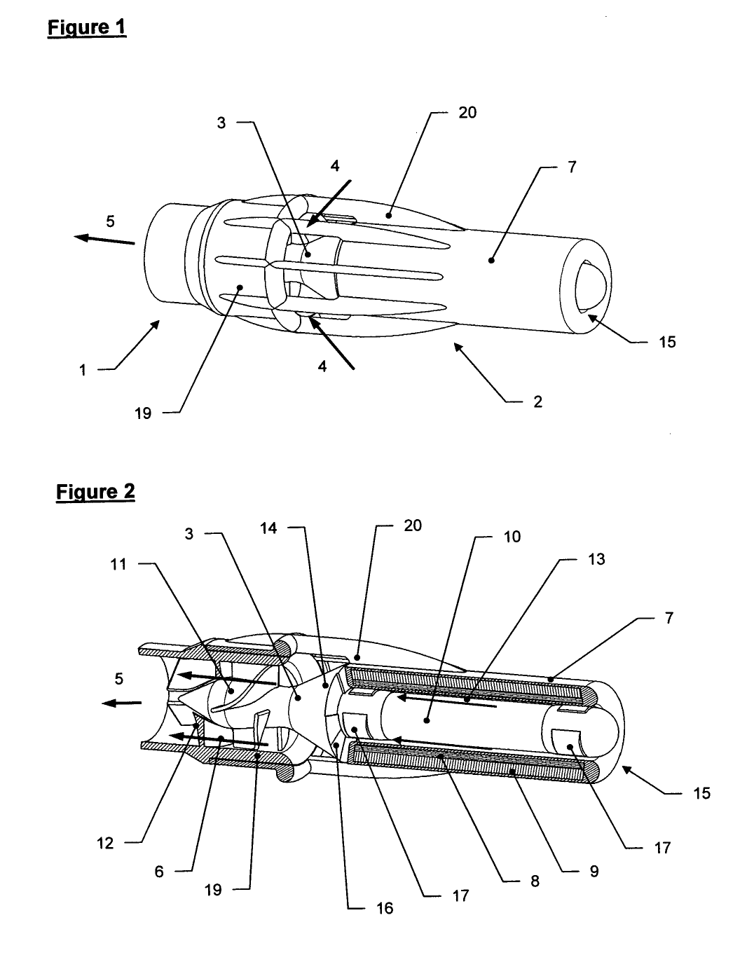

FIG. 1 is a perspective view of a first embodiment of a pump according to the invention;

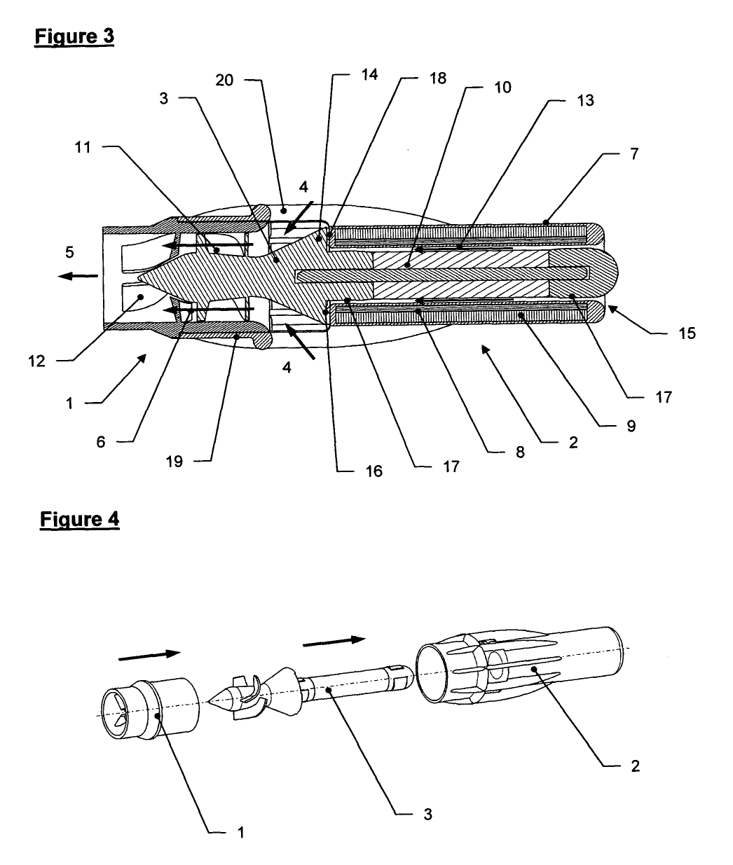

FIG. 2 is a perspective cutaway view of the pump of FIG. 1;

FIG. 3 is a full sectional view of the pump of FIG. 1;

FIG. 4 is an exploded view of the pump of FIG. 1;

FIG. 5 is a perspective cutaway view of a second embodiment of a pump according to the invention;

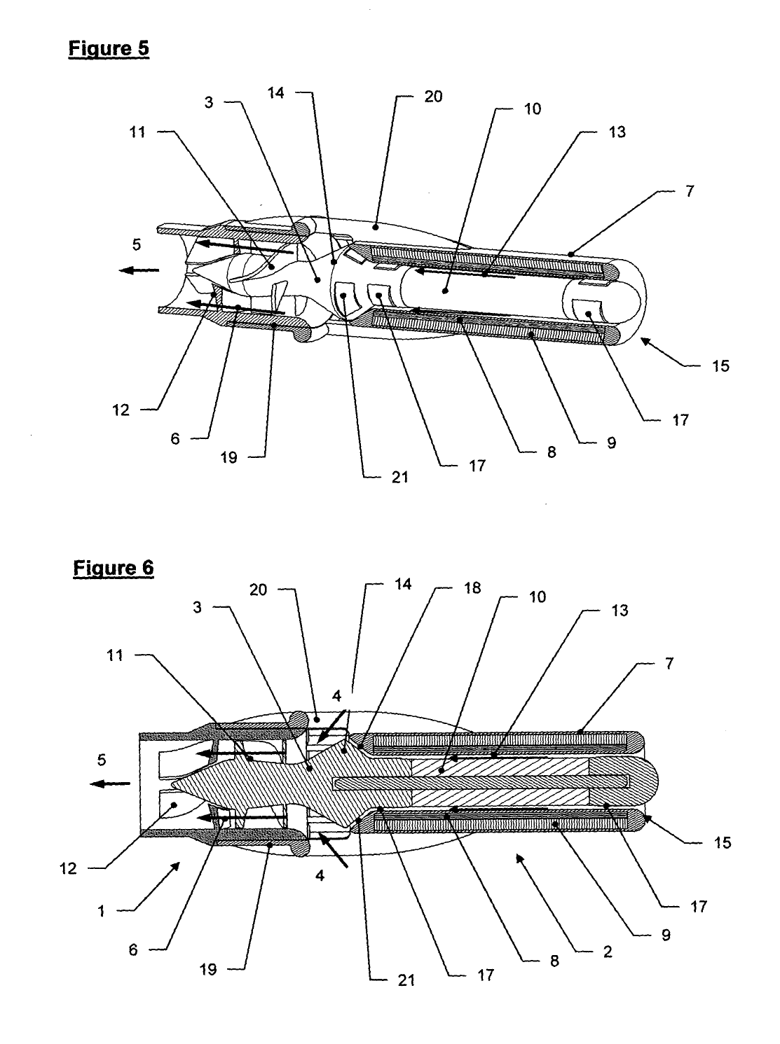

FIG. 6 is a full sectional view of the pump of FIG. 5;

FIG. 7 is a full sectional view of a third embodiment of a pump according to the invention;

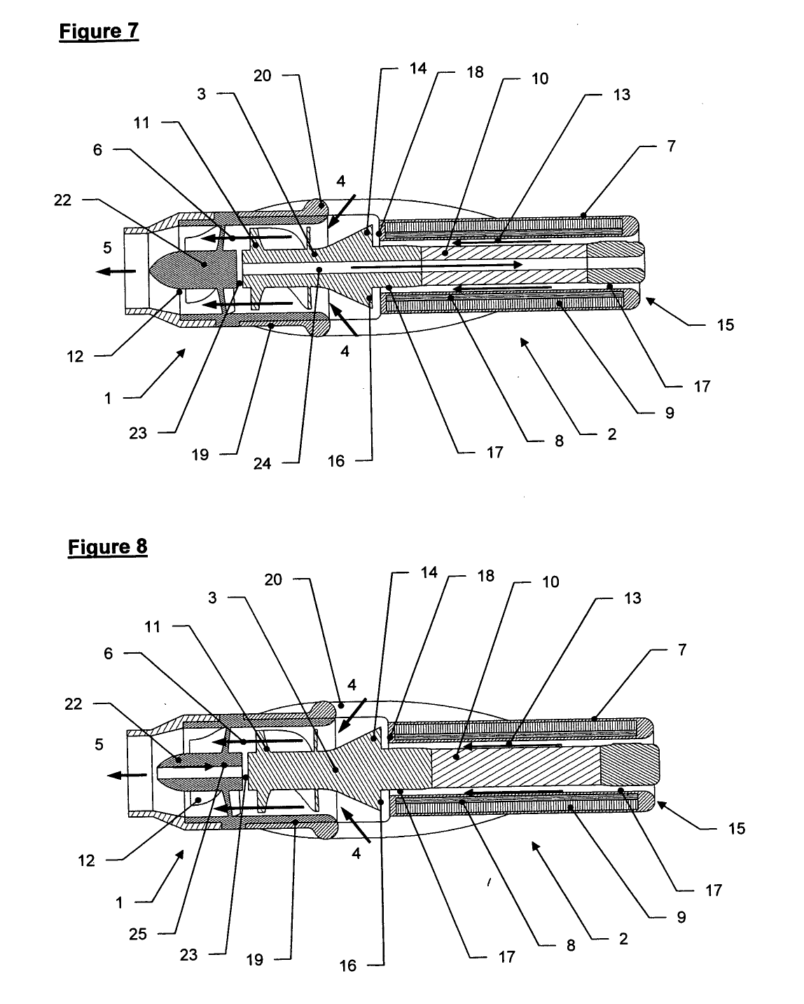

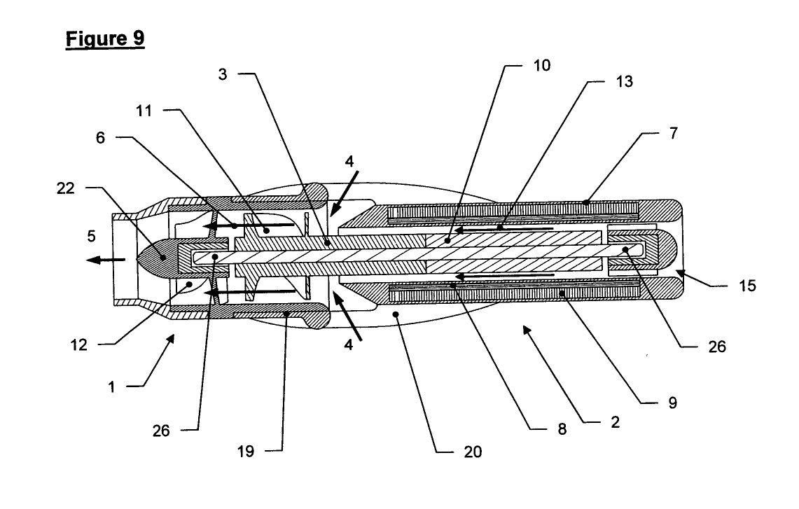

FIG. 8 is a full sectional view of a fourth embodiment of a pump according to the invention;

FIG. 9 is a full sectional view of a fifth embodiment of a pump according to the invention;

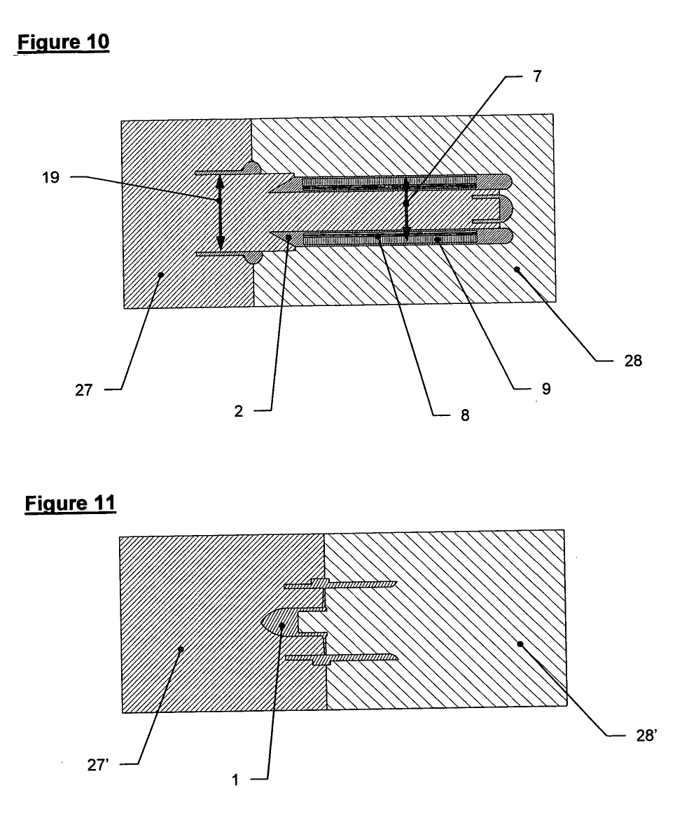

FIG. 10 is a schematic sectional view of exemplary tooling for making the tubular casing of a pump according to the invention; and

FIG. 11 is a further sectional view of such tooling, at right angles to the section of FIG. 10.

With reference to FIGS. 1 to 4, there is shown a miniature axial flow electric motor driven rotary pump for blood, which pump includes a front (downstream) longitudinally extending hollow tubular casing 1, a co-axial rear (upstream) longitudinally extending tubular casing 2, and a longitudinally extending rotatable element 3 which fits with a rotary clearance along the common axis of front casing 1 and rear casing 2. An inlet for blood 4 is provided in the side of the rear casing 2 and an outlet for blood 5 is provided in the end of the pump defined by the front casing 1. A primary blood flow path 6 is defined between the inlet 4 and outlet 5.

Integral with the rear casing 2 is a motor stator 7 comprising motor coils 8 and laminations 9. The rotatable element 3 includes of at least one motor magnet 10 that is arranged to co-operate with the motor coils 8.

The rotatable element 3 also includes an impeller 11 to create flow through the primary blood flow path 6. The front casing 1 includes a flow stator 12 to recover some of the whirl imparted to the blood flow by the impeller 11, thereby improving the efficiency of the pump.

In addition to the primary blood flow path, there is a defined secondary blood flow path 13 between the rotatable element 3 and an internal cylindrical surface of the rear casing 2, in a contactless arrangement which allows the pump to be near wearless in operation. The secondary blood flow path 13 is formed by a radial clearance between the internal cylindrical surface of the rear casing 2 and the rotatable element 3, and a circumferential clearance between an internal stepped surface 18 of the rear casing 2 and an annular flange 14 on the rotatable element 3.

An entrance to the secondary blood flow path 13 from the primary blood flow path is created by an open end 15 in the rear casing 2. An exit from the secondary blood flow path to the primary blood flow path is created by the clearance between the internal stepped surface 18 of the rear casing 2 and the annular flange 14 on the rotatable element 3.

In order to ensure that the secondary flow path 13 is able to effectively separate or space the rotatable element 3 from the front casing 1 and the rear casing 2, hydrodynamic bearing arrangements comprising axial hydrodynamic bearings 16 and radial hydrodynamic bearings 17 are provided in this embodiment. The hydrodynamic bearings also centralise the rotatable element 3 thereby preventing the latter from touching stationary parts of the pump.

The axial hydrodynamic bearings 16 are positioned on the annular flange 14 of the rotatable element 3 and act against the corresponding stepped surface 18 on the rear casing 2. Therefore the axial hydrodynamic bearings 16 are able to resist the thrust force generated by the impeller 11. As the pump only operates in one direction, and operates continuously, only a single direction axial hydrodynamic bearing 16 is required to axially stabilise the rotatable element 3.

The radial hydrodynamic bearings 17 are positioned in the radial clearance between the rotatable element 3 and the rear casing 2 and keep the rotatable element 3 centralised relative to stationary parts of the pump. Generally, the radial hydrodynamic bearings 17 should be spaced apart as far as possible to provide optimum centralisation.

Flow through the secondary blood flow path 13 is induced by the outlet residing in the low pressure area of the main pump inlet 4 such that blood is driven through the secondary flow path 13. If necessary, features such as small pumping vanes can be added to the secondary flow path 13 to increase flow rate through it.

The rear casing 2 comprises the previously described motor stator 7 and also a front annulus 19 that is integrally connected to the motor stator 7 by way of longitudinally extending connecting webs 20. The longitudinally extending gaps between the connecting webs 20 define the pump inlet 4 when the pump is fully assembled and also prevent the inlet 4 from exerting suction action against other structures of the heart. The inner diameter of the front annulus 19 can be of a larger diameter than the outer diameter of the motor stator section 7, which allows the rear casing 2 to be manufactured using low cost manufacturing techniques such as overmoulding.

With reference to FIG. 4, the pump is configured so that it is easy to assemble thereby reducing manufacturing costs. The rotatable element 3 is dropped into the rear casing 2 and retained by the front casing 1. The same applies to the second to fifth embodiments, which will now be described in more detail.

With reference to FIGS. 5 and 6, a second embodiment of the invention is shown. The second embodiment differs from the first embodiment in the region of the axial hydrodynamic bearing. In the first embodiment the axial hydrodynamic bearing 16 is perpendicular to the rotational axis of the rotatable element 3, whereas in the second embodiment an inclined or angled bearing 21 is used. This layout has the advantage that angled hydrodynamic bearing 21 has a self centralising ability when it is urged into the corresponding inclined face of the rear casing 2 by the thrust force of the impeller 11. Also, the secondary blood flow path 13 is smoother in the second embodiment.

All other features of the second embodiment are similar to those of the first embodiment.

With reference to FIG. 7, a third embodiment of the invention is shown. The third embodiment differs from the first and second embodiments by having a stationary hub 22 at the centre of the flow stator 12. The addition of a hub 22 in the flow stator 12 gives the potential for improved flow patterns to the benefit of, pump efficiency.

A possible problem with the stationary hub 22 might be that a gap 23 would be created between the hub 22 and the rotatable element 3, which gap could be liable to thrombus formation. To solve this problem, a central bore 24 is provided through the centre of the rotatable element 3 to allow blood to flow through the gap 23 and out through the open end 15 of the pump.

All other features of the third embodiment are similar to those of the previous embodiments.

With reference to FIG. 8, a fourth embodiment of the invention is shown. The fourth embodiment differs from the third embodiment by providing a central bore 25 in the stationary hub 22 as opposed to the central bore 24 in the rotatable element 3. The central bore 25 in the stationary hub 22 fulfils the same function as the central bore 24 in the rotatable element 3 of the third embodiment by allowing blood to flow through the gap 23 between the rotatable element 3 and the stator hub 22.

All other features of the fourth embodiment are similar to those of the previous embodiments.

With reference to FIG. 9, a fifth embodiment of the invention is shown. The fifth embodiment differs from previous embodiments by having the rotatable element 3 mounted with pivot bearings 26. The pivot bearings 26 are capable of resisting both axial and radial forces and therefore the annular flange 14, the axial hydrodynamic bearings 16 and radial hydrodynamic bearings 17 of the previous embodiments are not required. The stepped surface 18 on the rear casing 2 is also not required and the inlet 4 is therefore shaped for optimum streamlining.

All other features of the fifth embodiment are similar to those of the previous embodiments.

With reference to FIGS. 10 and 11, it will be described how the pump geometry as illustrated in the previous embodiments is amenable to manufacture by low cost manufacturing processes such as moulding.

With specific reference to the arrangement shown in FIG. 10, this shows the rear casing 2 in which the inner diameter of the front annulus 19 is of a larger diameter than the outer diameter of the motor stator section 7, which in turn allows the rear casing 2 to be easily formed in a moulding tool that comprises only a front mould tool half 27 and a rear mould tool half 28. As the connecting webs 20 do not at any point create a complete annulus these can be created by local voids in the rear tool half 28 (not shown), and there are no undercuts along the line of draw (or parting direction of the moulding tools). The motor coils 8 and motor laminations 9 can be encapsulated in the resulting unitary moulding by a conventional process, commonly known as overmoulding. The freedom from undercuts means that the relevant part can be formed in a simple two-part mould, without the need for specialist tool features such as collapsible cores.

FIG. 11 shows how the front casing 1 can also be formed a two piece moulding tool comprising a front tool half 27′ and a rear tool half 28′ in a similar way to that described above with reference to the rear casing 2 described above. Again, the moulding should be free of undercuts along the line of draw, and the resulting rear casing 1 can be fitted to the front casing as described above.

1. An axial flow rotary pump suitable for implantation into the human heart or vascular system, said pump comprising
(a) an elongate tubular casing (1,2) defining an inlet (4) for blood, an outlet (5) for blood longitudinally spaced from said inlet, and a primary substantially axial blood flow path (6) along the interior of the casing from said inlet to said outlet, said casing including an electric motor stator (7),
(b) an elongate rotatable element (3) arranged to fit within said casing with spacing between an outer surface of said rotatable element and an inner surface of said casing, said tubular rotatable element comprising an electric motor rotor portion (10) arranged to be driven by said electric motor stator; and a rotary impeller (11) for impelling blood along said blood flow path,
characterised in that the casing is formed as an upstream tubular member (2) having an open front end, and a downstream tubular member (1) having open front and rear ends, the upstream tubular member including the stator, and the downstream tubular member, which encircles the impeller, having a rear end fitted to the upstream tubular member in fluid tight manner.

2. A pump according to claim 1, wherein the downstream tubular element is a unitary moulding.

3. A pump according to claim 1 or 2, wherein the upstream tubular element comprises a unitary moulding encapsulating the stator.

4. A pump according to any of claims 1 to 3, wherein each of the tubular elements has a longitudinal axis of symmetry and/or is free of moulding undercuts.

5. A pump according to any of claims 1 to 4, wherein said rotatable element and said impeller together comprise a unitary moulding.

6. A pump according to any of claims 1 to 5, wherein the upstream tubular member has a mouth at its front end, said mouth being shaped to receive the rear end of the downstream tubular member.

7. A pump according to claim 6, wherein the downstream tubular member is a slide fit into said mouth, or said mouth has formations for complementary engagement with corresponding formations around the circumference of the rear end of the downstream tubular member.

8. A pump according to claim 7, wherein said downstream tubular element has a circumferential collar, to inhibit over-insertion thereof.

9. A pump according to any of claims 6 to 8, wherein the mouth at the front end of the upstream tubular member is of greater diameter than an opening at the rear end of the upstream tubular member.

10. A pump according to any of claims 6 to 9, wherein the mouth at the front end of the upstream tubular member is of greater diameter than an outer diameter of a rear end of the upstream member.

11. A pump according to any of claims 1 to 10, wherein the upstream tubular member has a series of circumferentially spaced inlets for blood around the periphery thereof.

12. A pump according to claim 11, wherein said inlets are separated from one another by a series of longitudinally extending ribs (20) extending from upstream of the inlets to downstream thereof.

13. A pump according to claim 12, wherein said ribs are provided with mechanical reinforcement which extends substantially around the circumference of the upstream tubular member.

14. A pump according to any of claims 1 to 13, wherein the rotatable element is provided with a circumferentially extending surface (16) which seats on a complementary circumferential surface (18) on the upstream tubular member.

15. A pump according to claim 14, wherein the complementary surfaces are approximately perpendicular to the axis of the rotatable element, or at an obtuse angle.
